(19) United States
(12) Patent Application Publication
(10) Pub. No.: US 2003/0021777 A1
(43) Pub. Date: Jan. 30, 2003

(54) PRODUCT COMPRISING BOUND FIBRINOGEN AND THROMBIN FOR USE IN WOUND THERAPY OR SURGICAL REPAIR

(76) Inventors: Roy Harris, Notts (GB); Sarah Margaret Middleton, Leics (GB)

Correspondence Address: Saliwanchik Lloyd & Saliwanchik, Suite A 1, 2421 N W 41st Street, Gainesville, FL, 32606-6669, US

(21) Appl. No.: 09/622,499

(22) PCT Filed: Feb. 22, 1999
(86) PCT No.: PCT/GB99/00533

(30) Foreign Application Priority Data
Feb. 20, 1998 (GB) 9803626.3
Aug. 18, 1998 (GB) 98180185.5

(51) Int. Cl. A61K 38/48

(52) U.S. Cl. 424/94.64

(57) ABSTRACT

A product comprises thrombin and microparticles having bound fibrinogen, as a combined preparation for simultaneous use in wound therapy or surgical repair. Another aspect lies in the use o insoluble microparticles having fibrinogen bound thereto, for the manufacture of a medicament for use in wound therapy or surgical repair of a patient having an abnormally low level of platelets.

### FIELD OF THE INVENTION

This invention relates to products comprising fibrinogen, especially microparticles having bound fibrinogen, and their therapeutic use. In particular, the invention relates to improvements in platelet substitutes and fibrin sealants.

### BACKGROUND OF THE INVENTION

A fibrin sealant is a biological adhesive composed of fibrinogen and thrombin. Such sealants are used extensively to assist wound healing and to provide sutureless closure of surgical wounds.

WO-A-9744015 describes the mechanism of action of a fibrin sealant, and in particular a composition comprising a dry mixture of soluble microparticles, respectively containing fibrinogen and thrombin, in free-flowing form. These microparticles are obtained by spray-drying.

Another fibrin sealant is disclosed in U.S. Pat. No. 4,427,651. This composition has freeze-dried components.

WO-A-9817319 discloses fibrinogen bound to microparticles. These products are proposed as platelet substitutes, and for use in the treatment of thrombocytopenia.

### SUMMARY OF THE INVENTION

The present invention is based, at least in part, on the observation that, when fibrinogen immobilised on an insoluble carrier is added to soluble fibrinogen and then thrombin is added, fibrin deposition is enhanced by comparison with the case in which the same amount of thrombin is added to each component separately. It appears that the immobilised fibrinogen may act as a nucleation site for fibrin formation.

According to a first aspect of the present invention, a product comprises thrombin and insoluble microparticles having bound fibrinogen, as a combined preparation for simultaneous use in wound therapy or surgical repair. In other words, the fibrinogen-bound insoluble microparticles enhance the utility of a fibrin sealant. They may replace some soluble fibrinogen (added or endogenous). Thus, they may be used instead of, or in addition to, a conventional soluble fibrinogen component of a fibrin sealant. A particular advantage of the present invention is that it allows the use of a fibrin sealant in circumstances where the patient has a low or zero platelet count, or a low level of fibrinogen (as in afibrinonaemia).

According to a second aspect of this invention, a platelet substitute comprising fibrinogen bound to insoluble microparticles may be functional in the absence of platelets, and can therefore be used in the treatment of patients where platelets are non-functional or absent, or are present at no more than a low level. It also indicates that, even when platelets are present, products of the type described in WO-A-9817319 will contribute to the procoagulant activity of the platelets by the enhancement of film formation, and interaction of fibrin with the GpI receptor on platelets, and hence the product will be more efficacious than previously thought. Accordingly, the present invention relates to the use of insoluble microparticles having fibrinogen bound thereto, for the manufacture of a medicament for use in wound therapy or surgical repair of a patient, and in particular a patient having an abnormally low level of platelets.

It has also been observed that fibrin can play the role of collagen, in producing procagulant activity in platelets. This reaction is brought about by fibrin binding through the platelets' GPIb receptor linking through vWF (von Willebrand's factor). This means that, in the presence of thrombin, fibrinogen-containing products may exert a procoagulant effect, including binding to GPIb through vWF. In addition, the products may also be capable of binding again through vWF to sub-endothelial collagen surfaces.

### DESCRIPTION OF THE INVENTION

All the respective components of a product of the present invention may be known. Their combination and their combined use are new. The amounts that will be used may be conventional, but can readily be determined according to the circumstances by one of ordinary skill in the art. The usual conditions will be taken into account, such as the nature and extent of the problem, the condition of the patient, and the desired effect.

Subjects that may be treated, according to the invention, are any requiring a fibrin sealant. Examples of patients having low platelet levels include cancer patients, e.g. following radiotherapy or chemotherapy, and patients who have been sensitised to blood-derived platelets. Other relevant conditions are idiopathic thrombocytopaenic purpura, thrombotic thrombocytopaenic purpura, aplastic anaemia, myelodysplastic syndromes, and Fanconi's syndrome.

The following Examples illustrate the invention (HSA is human serum albumin; Fg is fibrinogen).

### EXAMPLES

Following the procedure described in WO-A-9744015, microparticulate components of a fibrin sealant were prepared by spray-drying, from sucrose/fibrinogen (A) and sucrose/thrombin/HSA (B) mixtures. Similarly, fibrinogen-bound HSA microparticles (C) were prepared, as described in WO-A-9817319. Component C was vortexed prior to use, to avoid agglomeration.

Clot Strength Assay

A clot is formed by mixing the components in a plastic syringe. A clot formation time of 5 min is allowed. A bead is suspended in the syringe prior to the clot formation and the weight required to pull the bead through the formed clot is recorded.

### Example 1

The chosen ratio for the fibrin sealant was 30 mg fibrinogen:95 units thrombin. In order to achieve this ratio, aliquots of 222 mg A (sucrose/fibrinogen) were weighed into glass vials and dissolved in 1000 μl purified water. Aliquots of B (100 mg sucrose/thrombin) were dispensed into glass vials, and 500 Al purified water added. Eight further aliquots of each batch were prepared.

An aliquot of A was placed in the syringe via a pipette. The appropriate volume of C was added and the two solutions mixed by two uptakes of the pipette. An aliquot of B was then added, and the solutions mixed by three pipette uptakes.

Microcapsules (D) of human serum albumin (HSA), resuspended to give a final concentration equivalent to that of C (20 mg/ml protein, 51 mg/ml mannitol) were used as a control.

The results of the clot strength assay are given in Table 1.

TABLE 1

| Volume | Weight supported | Weight supported |
|---|---|---|
| added (μl) | by A/B + C (g) | by A/B + D (g) |
| 0 | 69.98 | 67.24 |
| 125 | 153.74 | 118.24 |
| 250 | 169.21 | 115.98 |
| 500 | 168.92 | 128.24 |
| 1000 | 84.29 | 94.19 |

The data reveal a significant increase in the clot strength upon addition to a A/B blend. The increase in clot strength observed upon addition of HSA microcapsules to a A/B blend suggests that there may be a bulking effect from the microcapsules which increases clot strength; however, there is a further increase in clot strength upon addition of C. The reduction in clot strength seen upon addition of the largest volume of both C and HSA microcapsules suggests that there is a volume effect: a stage may be reached where the total volume in the syringe is detrimental to clot formation.

### Example 2

In this Example, by contrast to Example 1, an investigation was made of the clots formed when other media such as purified water and 51 mg/ml mannitol solution were added to a A/B blend in comparison to those obtained with C. Accordingly, aliquots of A/B and C were prepared as described above, alongside blends with equivalent volumes of the following: 51 mg/ml mannitol (E); 20 mg/ml HSA and 51 mg/ml mannitol (F); and purified water (G). The results of the clot strength assay are given in Table 2.

TABLE 2

| | Weight | Weight | Weight | Weight | Weight |
|---|---|---|---|---|---|
| Volume | supported | supported | supported | supported | supported |
| added | by | by | by | by | by |
| (μl) | A/B + C(g) | A/B + D(g) | A/B + E(g) | A/B + F(g) | A/B + G(g) |
| 125 | 157.21 | 116.10 | 95.4 | 99.7 | 112.47 |
| 250 | 153.46 | 121.29 | 98.7 | 102.4 | 108.98 |
| 500 | 161.91 | 139.1 | 107.2 | 115.7 | 140.29 |
| 1000 | 79.10 | 101.28 | 137.2 | 124.3 | 99.74 |

The data reveal a significant enhancement of clot strength upon the addition of C. The clot strength observed for A/B with additional water is also greater than that seen for A/B alone (compare 112.47 g with the value of 70 g from Table 1), suggesting that the clot strength is dependant on the volume in the syringe. Again, it was noted that increasing the volume of C over 125 μl has no significant effect on clot strength.

### Example 3

Commercial information reveals Centeon Fibrin Sealant to contain 60-115 mg/ml fibrinogen, 400-600 units/ml thrombin, 900-1100 KI units/ml aprotinin and 40-80 units/ml Factor XIII. A freeze-dried preparation was prepared which mimicked the ratio of 1:5.55 (fibrinogen:thrombin) described above. Fibrinogen was reconstituted using 50 ml purified water which resulted in a fibrinogen concentration of 26 mg/ml. A vial of freeze-dried thrombin containing 1000 units was reconstituted in 6.9 ml calcium chloride solution (40 mM).

The desired volumes of C were centrifuged and the supernatants discarded; the pellets were then reconstituted in 1 ml fibrinogen solution. The 1 ml sample was then placed in the syringe via pipette. A 1 ml aliquot of the thrombin solution was then pipetted into the syringe, and final mixing was performed by one uptake of the pipette. Five minutes of clotting time was allowed before the weight supported by the resultant clot was determined. The results of the clot strength assay are given in Table 3.

TABLE 3

| Volume C added to A/B (μl) | Weight supported (g) |
|---|---|
| 0 | 94.92 |
| 25 | 93.27 |
| 50 | 116.66 |
| 75 | 121.01 |
| 100 | 128.94 |
| 125 | 134.09 |
| 150 | 149.43 |

The results obtained reveal a relationship between the level of C and the strength of the formed clot.

Further experiments have shown that the clot strength is substantially maintained after longer clotting times, e.g. up to 4 hours.

### Example 4

Aliquots of C (50, 100 and 150 μl) were centrifuged at 10,000 rpm for 5 min. The aliquots equated to 250, 500 and 750 ng immobilised fibrinogen, respectively.

The pellets were each reconstituted in 500 μl of the fibrinogen solution (26 mg/ml, Haemocomplettan). This was then mixed with 500 ,1 thrombin solution (100 units/ml).

Adhesive strength was measured by the weight required to separate two pieces of tissue bonded together. The results are given in Table 4.

TABLE 4

| Volume of C | Fg Immobilised | Weight Supported | Adhesive Strength |
|---|---|---|---|
| (μl) | (ng) | (g) | (mg/mm²) |
| 50 | 250 | 160.3 | 173 |
| 100 | 500 | 169.4 | 183 |
| 150 | 750 | 177.3 | 192 |

The addition of C appears to increase the adhesive strength to the same magnitude as seen for the clot strength assays (50%).

### Example 5

In this Example, the amount of fibrinogen provided by A was varied, at a constant thrombin concentration of 100 units. The blends were assessed for clot strength with and without the addition of C (150 μl, 750 ng immobilised Fg). 12 aliquots of A were weighed into glass vials, to provide 5, 10, 15, 20, 25 and 30 mg as required Fg weights, in duplicate. For example, 5 mg Fg corresponds to 35 mg A (14.3 mg Fg/100 mg product).

12 vials containing 100 mg B were also prepared. 1 vial of C was thawed and vortexed thoroughly. 6 aliquots of 150 μl were then removed and centrifuged (5 min at 10,000 rpm). The supernatants were removed and discarded.

Each of the thrombin aliquots was dissolved in 500 μl purified water. Each of the fibrinogen aliquots was dissolved in 1 ml purified water. The samples required for the C investigation were taken (6) and each of the fibrinogen components used to reconstitute the pellets. As a control, the effect of variable fibrinogen levels on the clot strength was measured. The results are given in Table 5.

TABLE 5

| | |
|---|---|
| Mass of Fibrinogen in Blend | Weight Supported by Clot (g) |

| (mg) | A | A + C |
|---|---|---|
| 0 | 0 | 0 |
| 5 | 10.99 | 10.99 |
| 10 | 23.36 | 45.84 |
| 15 | 35.87 | 58.72 |
| 20 | 49.82 | 104.73 |
| 25 | 79.48 | 127.21 |
| 30 | 100.02 | 154.64 |

The results show that the level in a fibrin sealant blend (of fibrinogen) can be significantly (40-50%) reduced, and provide the same clot strength as exhibited by the optimum ratio (30 mg Fg: 100 units thrombin). This is important commercially, and provides a degree of control over clotting time and resultant clot strength.

### Example 6

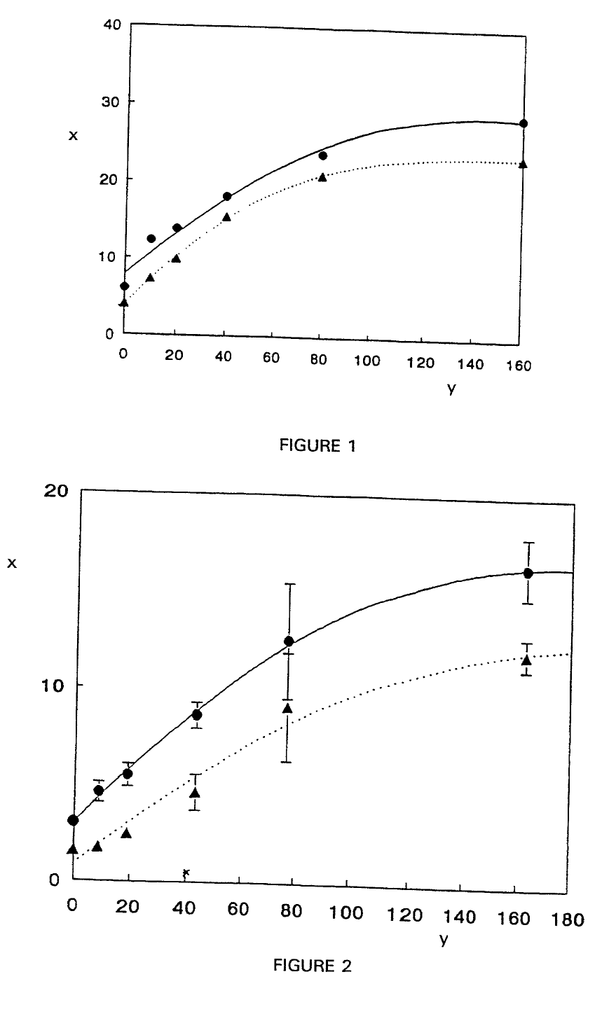

This Example provides evidence of the utility of C alone. Experiments were conducted in the absence of platelets in a perfusion chamber at low and high shear rates, and the results are shown in FIGS. 1 (shear rate 1600/sec) and 2 (shear rate 300/sec). The degree of coverage (x, %) was plotted against total platelets (y, g/l), using C and also, as a control, HSA microcapsules with no bound fibrinogen. In each case, the control is represented as a dotted line. The results show an increase in % of coverage using C, by comparison with control (HSA microcapsules with no bound fibrinogen).

1. A product comprising thrombin and microparticles having bound fibrinogen, as a combined preparation for simultaneous use in wound therapy or surgical repair.

2. A product according to claim 1, for use in a patient having an abnormally low level of platelets.

3. A product according to claim 1 or claim 2, comprising soluble microparticles comprising thrombin, soluble microparticles comprising fibrinogen and insoluble microparticles having fibrinogen bound thereto.

4. Use of insoluble microparticles having fibrinogen bound thereto, for the manufacture of a medicament for use in wound therapy or surgical repair of a patient having an abnormally low level of platelets.

5. Use according to claim 4, wherein the patient has cancer, and has undergone radiotherapy or chemotherapy.

6. Use according to claim 4, wherein the patient has been sensitised to blood-derived platelets.
